(19) United States
(12) Patent Application Publication
(10) Pub. No.: US 2010/0121142 A1
(43) Pub. Date: May 13, 2010

(54) Minimally Invasive Imaging Device

(76) Inventors: Xiaolong OuYang, Palo Alto, CA (US); James S. Cybulski, Menlo Park, CA (US); Fred R. Seddiqui, Los Altos, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 12/269,772

(22) Filed: Nov. 12, 2008

(51) Int. Cl. A61B 1/04

(52) U.S. Cl. 600/109; 600/104

(57) ABSTRACT

Aspects of the invention include minimally invasive internal target tissue site imaging devices. Devices according to embodiments of the invention include an elongate member dimensioned to access an internal target tissue location, e.g., an intervertebral disc, and having a proximal end and a distal end; a white light source at the distal end; a near-infra-red light source the distal end; and an imaging sensor at the distal end. Also provided are methods of using the systems in imaging applications, as well as kits for performing the methods.

Many pathological conditions in the human body may be caused by enlargement, movement, displacement and/or a variety of other changes of bodily tissue, causing the tissue to press against (or “impinge on”) one or more otherwise normal tissues or organs. For example, a cancerous tumor may press against an adjacent organ and adversely affect the functioning and/or the health of that organ. In other cases, bony growths (or “bone spurs”), arthritic changes in bone and/or soft tissue, redundant soft tissue, or other hypertrophic bone or soft tissue conditions may impinge on nearby nerve and/or vascular tissues and compromise functioning of one or more nerves, reduce blood flow through a blood vessel, or both. Other examples of tissues which may grow or move to press against adjacent tissues include ligaments, tendons, cysts, cartilage, scar tissue, blood vessels, adipose tissue, tumor, hematoma, and inflammatory tissue.

The intervertebral disc is composed of a thick outer ring of cartilage (annulus) and an inner gel-like substance (nucleus pulposus). A three-dimensional view of an intervertebral disc is provided in FIG. 1. The annulus contains collagen fibers that form concentric lamellae that surround the nucleus and insert into the endplates of the adjacent vertebral bodies. The nucleus pulposus comprises proteoglycans entrapped by a network of collagen and elastin fibers which has the capacity to bind water. When healthy, the intervertebral disc keeps the spine flexible and serves as a shock absorber by allowing the body to accept and dissipate loads across multiple levels in the spine.

With respect to the spine and intervertebral discs, a variety of medical conditions can occur in which it is desirable to ultimately surgically remove at least some of if not all of an intervertebral disc. As such, a variety of different conditions exist where partial or total disc removal is desirable.

One such condition is disc herniation. Over time, the nucleus pulposus becomes less fluid and more viscous as a result of age, normal wear and tear, and damage caused from an injury. The proteoglycan and water from within the nucleus decreases which in turn results in the nucleus drying out and becoming smaller and compressed. Additionally, the annulus tends to thicken, desiccate, and become more rigid, lessening its ability to elastically deform under load and making it susceptible to disc fissures.

A fissure occurs when the fibrous components of the annulus become separated in particular areas, creating a tear within the annulus. The most common type of fissure is a radial fissure in which the tear is perpendicular to the direction of the fibers. A fissure associated with disc herniation generally falls into three types of categories: 1) contained disc herniation (also known as contained disc protrusion); 2) extruded disc herniation; and 3) sequestered disc herniation (also known as a free fragment.) In a contained herniation, a portion of the disc protrudes or bulges from a normal boundary of the disc but does not breach the outer annulus fibrosis. In an extruded herniation, the annulus is disrupted and a segment of the nucleus protrudes/extrudes from the disc. However, in this condition, the nucleus within the disc remains contiguous with the extruded fragment. With a sequestered disc herniation, a nucleus fragment separates from the nucleus and disc.

As the posterior and posterolateral portions of the annulus are most susceptible to herniation, in many instances, the nucleus pulposus progresses into the fissure from the nucleus in a posteriorly or posterolateral direction. Additionally, biochemicals contained within the nucleus pulposus may escape through the annulus causing inflammation and irritating adjacent nerves. Symptoms of a herniated disc generally include sharp back or neck pain which radiates into the extremities, numbness, muscle weakness, and in late stages, paralysis, muscle atrophy and bladder and bowel incontinence.

Conservative therapy is the first line of treating a herniated disc which includes bed rest, medications to reduce inflammation and pain, physical therapy, patient education on proper body mechanics and weight control.

If conservative therapy offers no improvement then surgery is recommended. Open discectomy is the most common surgical treatment for ruptured or herniated discs. The procedure involves an incision in the skin over the spine to remove the herniated disc material so it no longer presses on the nerves and spinal cord. Before the disc material is removed, some of the bone from the affected vertebra may be removed using a laminotomy or laminectomy to allow the surgeon to better see the area. As an alternative to open surgery, minimally invasive techniques have been rapidly replacing open surgery in treating herniated discs. Minimally invasive surgery utilizes small skin incisions, thereby minimizing the damaging effects of large muscle retraction and offering rapid recovery, less post-operative pain and small incisional scars.

### SUMMARY

Aspects of the invention include minimally invasive imaging devices. Devices according to embodiments of the invention include an elongate member dimensioned to access an internal tissue target site, e.g., an intervertebral disc target site, and having a proximal and distal end; a white light source at the distal end; a near-infra-red light source at the distal end; and an imaging sensor at the distal end. Also provided are methods of using the devices and systems that include the same in imaging applications, as well as kits for performing the methods.

### BRIEF DESCRIPTION OF THE FIGURES

FIG. 1 provides a three-dimensional view of an intervertebral disc according to one embodiment of the invention.

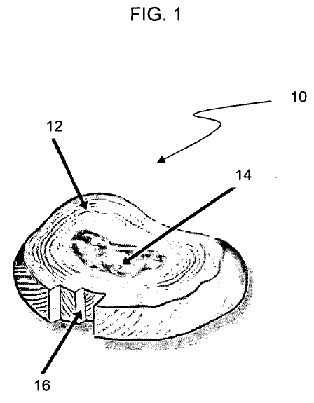

FIG. 2 provides a view of a cross section of the proximal end of a surgical device configured to remove the nucleus pulposus of an intervertebral disc (IVD) according to an embodiment of the invention.

FIG. 3 provides a schematic of the operational framework of a processor that may be present in a device according to embodiments of the invention.

FIG. 4 illustrates a visualization device according to one embodiment of the invention viewing the nucleus pulposus of an intervertebral disc through an access port provided by a access device, such as a cannula.

### DETAILED DESCRIPTION

Aspects of the invention include imaging devices of minimally invasive surgical device. Devices according to embodiments of the invention include an elongate member dimensioned to access an internal tissue target site, e.g., an intervertebral disc target site, and having a proximal and distal end; a white light source at the distal end; a near-infra-red light source at the distal end; and an imaging sensor at the distal end. Also provided are methods of using the devices and systems that include the same in imaging applications, as well as kits for performing the methods.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

In further describing various aspects of the invention, embodiments of the minimally invasive imaging devices are reviewed first in greater detail, followed by a review of embodiments of methods of using the devices.

### Minimally Invasive Imaging Device

As summarized above, aspects of the invention include minimally invasive imaging devices. The imaging devices of the invention are minimally invasive, such that they may be introduced to an internal target site of a patient, e.g., a location of an intervertebral disc, through a minimal incision, e.g., one that is less than the size of an incision employed for an access device having a outer diameter of 20 mm or larger, e.g., less than 75% the size of such an incision, such as less than 50% of the size of such an incision, or smaller.

The devices include an elongate member having a proximal end and a distal end. As this component of the device is elongate, it has length that is 1.5 times or longer than its width, such as 2 times or longer than its width, including 5 or even 10 times or longer than its width, e.g., 20 times longer than its width, 30 times longer than its width, or longer.

The elongate member is dimensioned to access an internal target tissue site. The elongate member may be dimensioned to access a variety of different internal target tissue sites, depending on the purpose for which it is designed. For convenience only, the present invention is described further primarily in terms of embodiments that are dimension to image an intervertebral disc. However, the invention is not limited to intervertebral disc imaging devices.

By “dimensioned to access an intervertebral disc” is meant that that, at least the distal end of the device has a longest cross-sectional dimension, e.g., outer diameter, that is 10 mm or less, such as 6 mm or less and including 3 mm or less, where in certain embodiments the longest cross-sectional dimension has a length ranging from 1 to 10 mm, such as 2 to 10 mm and including 3 to 5 mm. In certain instances, the device has a longest cross-sectional dimension that is 10 mm or less, such as 8 mm or less and including 7 mm or less, where in certain embodiments the longest cross-sectional dimension has a length ranging from 5 to 10 mm, such as 6 to 9 mm, and including 6 to 8 mm. The elongate member may be solid or include one or more lumens, such that it may be viewed as a catheter. The term “catheter” is employed in its convention sense to refer to a hollow, flexible tube configured to be inserted into a body. Catheters of the invention may include a single lumen, or two or more lumens, e.g., three or more lumens, etc, as desired. Depending on the particular embodiment, the elongate members may be flexible or rigid, articulated or steerable, and may be fabricated from any convenient material.

Located at the distal end of the elongate member is an imaging sensor and two or more spectrally distinct light sources. By “located at the distal end” is meant that the item of interest (e.g., the imaging sensor, the light sources) is present at the distal end of the elongate member, or near the distal end of the elongate member, e.g., within 50 mm or closer to the distal end, such as within 25 mm or closer to the distal end and including within 10 mm or closer to the distal end, including at the distal end. Where desired, the near-infra-red and white light illumination sources may be positioned to cover the same field of view (FOV) as the imaging device, e.g., CMOS sensor, which in turn may be positioned to visualize with the area at which the dissection tool is directed, e.g., as described in greater detail below. The image sensor may be on the same plane of the illumination elements, or different, as desired.

The light sources may be integrated with the elongate member such that they are configured relative to the elongate member such that the light source element cannot be removed from the remainder of the elongate member without significantly compromising the structure of the elongate member. As such, the integrated illumination element of these embodiments is not readily removable from the remainder of the elongate member, such that the illumination element and remainder of the elongate member form an inter-related whole.

Imaging sensors of interest are miniature in size so as to be positionable at the distal end of the elongate member. Miniature imaging sensors of interest are those that, when integrated at the distal end of an elongated structure along with an illumination source, e.g., such as an LED as reviewed below, can be positioned on a probe having a longest cross section dimension of 6 mm or less, such as 5 mm or less, including 4 mm or less, and even 3 mm or less. In certain embodiments, the miniature imaging sensors have a longest cross-section dimension (such as a diagonal dimension) of 5 mm or less, such 3 mm or less, where in certain instances the sensors may have a longest cross-sectional dimension ranging from 2 to 3 mm. In certain embodiments, the miniature imaging sensors have a cross-sectional area that is sufficiently small for its intended use and yet retain a sufficiently high matrix resolution. Certain imaging sensors of the invention have a cross-sectional area (i.e. an x-y dimension, also known as packaged chip size) that is 2 mm×2 mm or less, such as 1.8 mm×1.8 mm or less, and yet have a matrix resolution of 400×400 or greater, such as 640×480 or greater. Imaging sensors of interest are those that include a photosensitive component, e.g., array of photosensitive elements, coupled to an integrated circuit, where the integrated circuit is configured to obtain input signals from the photosensitive array and output a signal. The image sensors of interest may be viewed as integrated circuit image sensors, and include complementary metal-oxide-semiconductor (CMOS) sensors or charge-coupled device (CCD) sensors. The image sensors may further include a lens positioned relative to the photosensitive component so as to focus images on the photosensitive component. A signal conductor may be present to connect the image sensor at the distal and to a device at the proximal end of the elongate member, e.g., in the form of one or more wires running along the length of the elongate member from the distal to the proximal end. Imaging sensors of interest include, but are not limited to, those obtainable from: OminiVision Technologies Inc., Sony Electronics Corporations, Cypress Semiconductors. The imaging sensors may be integrated with the elongated structure. As the imaging sensor(s) is integrated at the distal end of the elongated structure, it cannot be removed from the remainder of the elongated structure without significantly compromising the structure of elongated structure. As such, the integrated visualization element is not readily removable from the remainder of the elongated structure, such that the visualization element and remainder of the elongated structure form an inter-related whole.

While any convenient imaging sensor may be employed in devices of the invention, in certain instances the imaging sensor is a CMOS sensor. Of interest as CMOS sensors are the OmniPixel line of CMOS sensors available from OmniVision (Sunnyvale, Calif.), including the OmniPixel, OmniPixel2, OmniPixel3, OmniPixel3-HS and OmniBSI lines of CMOS sensors. These sensors may be either frontside or backside illumination sensors, and have sufficiently small dimensions while maintained sufficient functionality to be positioned at the distal end of the minimally invasive devices of the invention. Aspects of these sensors are further described in one or more the following U.S. patents, the disclosures of which are herein incorporated by reference: U.S. Pat. Nos. 7,388,242; 7,368,772; 7,355,228; 7,345,330; 7,344,910; 7,268,335; 7,209,601; 7,196,314; 7,193,198; 7,161,130; and 7,154,137.

In addition to the imaging sensor, two or more spectrally distinct light sources are also present at the distal end of the elongated member. By spectrally distinct is meant that the light sources emit light at wavelengths that do not substantially overlap. Of interest are “white light” light sources and near-infra-red light sources. “White light” light sources are those light sources which are configured to illuminate a tissue location with white light, i.e., electromagnetic radiation of a wavelength that is visible to the human eye (about 400-700 nm), or up to 380-750 nm. Near-Infra-red light sources are sources of light which are configured to illuminate a tissue location with near-infra-red light, i.e., near infra-red radiation having wavelengths between about 700 nm and 1100 nm. The light sources may be light emitting diodes configured to emit light of the desired wavelength range, or optical conveyance elements, e.g., optical fibers, configured to convey light of the desired wavelength range from a location other than the distal end of the elongate member, e.g., a location at the proximal end of the elongate member, to the distal end of the elongate member. Examples of a “white light” light source include a white light emitting diode and an optical fiber operatively coupled to a white light emitter located at the proximal end of the catheter. Examples of an infra red light source include an infra red emitting diode and an optical fiber operatively coupled to an infra red light emitter located at the proximal end of the catheter. As with the image sensors, the light sources may include a conductive element, e.g., wire, optical fiber, which runs the length of the elongate member to provide for control of the light sources from a location outside the body, e.g., an extracorporeal control device. In certain embodiments, the light sources may be configured to communicate wirelessly with an extracorporeal control device.

In certain embodiments, the imaging device further includes a tissue modifier. Tissue modifiers are components or sub-devices that interact with tissue in some manner to modify the tissue in a desired way. The term modify is used broadly to refer to changing in some way, including cutting the tissue, ablating the tissue, delivering an agent(s) to the tissue, freezing the tissue, etc. As such, of interest as tissue modifiers are tissue cutters, tissue ablators, tissue freezing/heating elements, agent delivery devices, etc. Tissue cutters of interest include, but are not limited to: blades, liquid jet devices, lasers and the like. Tissue ablators of interest include, but are not limited to ablation devices, such as devices for delivery ultrasonic energy (e.g., as employed in ultrasonic ablation), devices for delivering plasma energy, devices for delivery radiofrequency (RF) energy, devices for delivering microwave energy, etc. Energy transfer devices of interest include, but are not limited to: devices for modulating the temperature of tissue, e.g., freezing or heating devices, etc.

In certain embodiments, the imaging device may further include one or more lumens that run at least the substantial length of the device, e.g., for performing a variety of different functions. In certain embodiments where it is desired to flush (i.e., wash) the location of the target tissue at the distal end of the elongate member (e.g., to remove cut tissue from the location, etc.), the elongate member may include both an irrigation and aspiration lumen. During use, the irrigation lumen is operatively connected to a fluid source (e.g., physiologically acceptable fluid such as saline) at the proximal end of the device, where the fluid source is configured to introduce fluid into the lumen under positive pressure, e.g., at a pressure ranging from 0 to 500 mm Hg so that fluid is conveyed along the irrigation lumen and out the distal end. While the dimensions of the irrigating lumen may vary, in certain embodiments the longest cross-sectional dimension of the irrigation lumen ranges from 1 to 3 mm. During use, the aspiration lumen is operatively connected to a source of negative pressure (e.g., vacuum source) at the proximal end of the device, where the negative pressure source is configured to draw fluid from the tissue location at the distal end into the irrigation lumen under positive pressure, e.g., at a pressure ranging from 50 to 600 mm Hg, so that fluid is removed from the tissue site and conveyed along the irrigation lumen and out the proximal end, e.g., into a waste reservoir. While the dimensions of the aspiration lumen may vary, in certain embodiments the longest cross-sectional dimension of the aspiration lumen ranges from 2 to 4 mm, such as 2 to 3 mm.

In certain embodiments, the imaging devices of the invention are used in conjunction with a controller configured to control illumination of the illumination elements and/or capture of images (e.g., as still imaged or video output) from the image sensors. This controller may take a variety of different formats, including hardware, software and combinations thereof. The controller may be physically located relative to the device at any convenient location, where the controller may be present at the distal end of the device, at some point between the distal and proximal ends or at the proximal end of the device. In certain embodiments, the controller may be distinct from the device, such the device includes a controller interface for operatively coupling to the distinct controller, or the controller may be integral with the device.

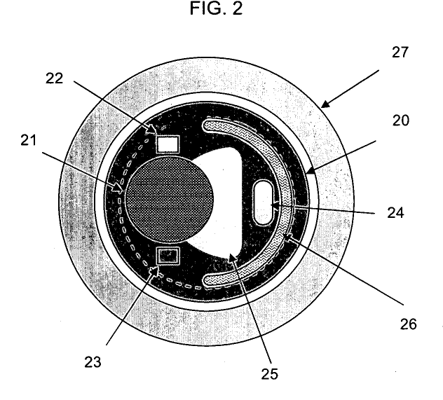

FIG. 2 provides a cross-sectional view of the distal end of an imaging device according to one embodiment of the invention, where the imaging device is configured to be employed in the surgical removal of the nucleus pulposus of an intervertebral disc. As such, the device shown in FIG. 2 is actually a surgically device which has imaging functionality according to the invention integrated into it. In FIG. 2, distal end of elongated member 20 (in this embodiment a catheter) includes imaging sensor 21 (labeled camera w/lens), as well as a white light source 22 which is a white light LED and a near-infra-red light source 23 which is a near-infra-red LED. Also shown is an irrigation lumen 24 and aspiration lumen 25. In addition, the device includes a tissue modifier in the form of a dissection electrode 26 (where the modifier may be any desired type of modifier, such as ultrasound, RF, purely mechanical modifier, etc., e.g., as elaborated on further elsewhere in this disclosure). Also shown is the access device 27 which is in the form of a introducer tube or sheath, as described in greater detail below.

In certain embodiments, the controller (when present) is configured to alternate illumination of the target tissue, e.g., an intervertebral disc or portion thereof, with the white light source and the near-infra-red source. By alternate is meant that at some point there is a switch from illumination with the white light source and illumination with the near-infra-red light source. In these embodiments, the controller may also be configured to cause the image sensor(s) to obtain one or more images, e.g., stills or video, under each type of illumination, e.g., so as to obtain white light image data and infrared image data. The phrase “image data” refers to data that can be used by a processor to produce some type of human viewable image e.g., a still image or a video, on an appropriate display medium, e.g., a monitor.

In certain embodiments, the processor is configured to provide to a user multi-spectral image that is produced from image data obtained under white light illumination and near-infra-red illumination. The multi-spectral image may be generated to provide to a user a variety of different types of information not available to a user with image data obtained under a single spectra of illumination. For example, the multi-spectral image may be generated to provide a user with a three-dimensional effect that presents depth information to the user during use, e.g., during tissue dissection, irrigation and aspiration.

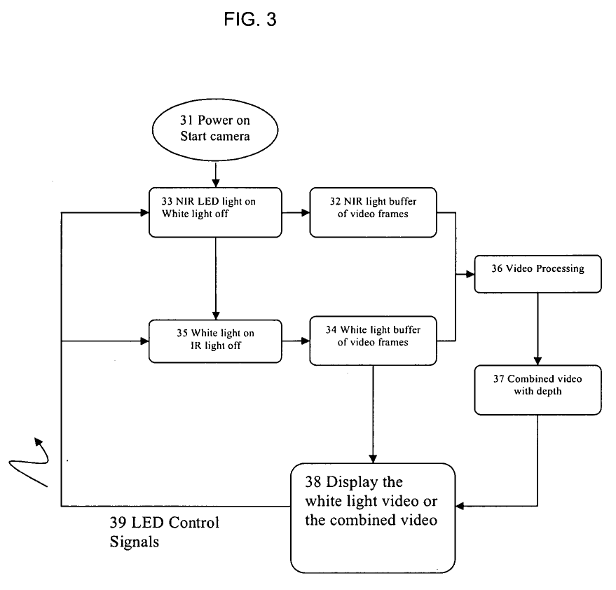

In certain embodiments, the processor may be configured to produce a video from the image data that is obtained under white light, under near-infra-red light or a combination of data taken under illumination of both kinds of light, i.e., to produce a multi-spectral or combined video. For example, if the target tissue site is relatively free of fluid, a user may desire to view the site under white light illumination. Alternatively, where the target tissue site is filled with fluid, a user may desire to view the site under near-infra-red illumination. As illustrated in FIG. 3, following power on 31 of the camera, the camera obtains an NIR light buffer of video frames 32 with the NIR LED on and the white light off 33. In addition, the camera obtains an white light buffer of video frames 34 with the NIR LED off and the white light on 35. The resultant video data is processed at step 36 to produce combined video with depth 37. At step 38, a user is given a choice of viewing a white light video obtained solely under white light illumination, or a combined video of a video obtained under white light illumination and a video obtained under near-infra-red illumination. By combined video is meant a video image that incorporates image data obtained under both white light illumination and near-infra-red illumination. Though not shown in FIG. 3, the processor may also be configured to provide a user with a choice of viewing only near-infra-red image data. User choice may be employed to control LEDs, as illustrated at step 39.

The above processor and image display functionalities may be physically implemented by any convenient combination of hardware and software. The devices may be operated according to any convenient algorithm. In certain instances, normal mode provides image data to a user in the form of white LED videos. However, when the target tissue site is flooded or obscured, near-infra-red LED mode is turned on by pushing a button. Where desired, both the sensor settings and the video processing settings may be switched adaptively.

The devices or components thereof may be configured for one time use (i.e., disposable) or re-usable, e.g., where the components are configured to be used two or more times before disposal, e.g., where the device components are sterilizable.

### Methods

Aspects of the invention further include methods of imaging an internal tissue site with imaging devices of the invention. A variety of internal tissue sites can be imaged with devices of the invention. In certain embodiments, the methods are methods of imaging an intervertebral disc in a minimally invasive manner.

With respect to imaging an intervertebral disc or portion thereof, e.g., exterior of the disc, nucleus pulposus, etc., embodiments of such methods include positioning a distal end of a minimally invasive intervertebral disc imaging device of the invention in viewing relationship to an intervertebral disc or portion of there, e.g., nucleus pulposus. By viewing relationship is meant that the distal end is positioned within 40 mm, such as within 5 mm, of the target tissue site of interest. Positioning the distal end in viewing relation to the desired target tissue may be accomplished using any convenient approach, including through use of an access device, such as a cannula, which may or may not be fitted with a trocar, as desired. Following positioning of the distal end of the imaging device in viewing relationship to the target tissue, the target tissue, e.g., intervertebral disc or portion thereof, is illuminated with one of the white light source and the near-infra-red light source; and image data is obtained with an image sensor, e.g., in the form of capturing one or more image frames of the illuminated intervertebral disc with the imaging sensor. In certain embodiments, the methods include sequentially illuminating the target tissue with the white light source and the near-infra-red light source. By sequentially illuminating is meant that a first of the light sources is employed and then a second of the light sources is employed. In those embodiments where the target tissue is sequentially illuminated by the light sources, there may or may not be a rest time when no illumination takes place between illumination with the different types of light sources. Where such a rest time is employed, the length of a given rest time may vary, and in certain instances is less than 1 sec. The length of any illumination period for any of the illumination sources may vary from being always on for extended periods of time to being on at defined time intervals, e.g., where the illumination elements are alternately on at 0.5 sec intervals or less.

Image data obtained according to the methods of the invention is output to a user in the form of an image, e.g., using a monitor or other convenient medium as a display means. In certain embodiments, the image is a still image, while in other embodiments the image may be a video.

In certain embodiments, the methods include capturing a white light video of the intervertebral disc made up of image frames obtained under white light illumination and capturing a near-infra-red light video of the intervertebral disc made up of image frames obtained under near-infra-red illumination. Where desired, the methods may include producing a combined video made up of components of the white light video and near-infra-red video, e.g., to provide a user with multi-spectral images of the target tissue, e.g., to provide the user with a three-dimensional type view. Where desired, the method includes providing to a user a choice of viewing the white light video and the combined video.

In certain embodiments, the methods include a step of tissue modification in addition to the tissue viewing. For example, the methods may include a step of tissue removal, e.g., using a combination of tissue cutting and irrigation or flushing. For example, the methods may include cutting a least a portion of the tissue and then removing the cut tissue from the site, e.g., by flushing at least a portion of the imaged tissue location using a fluid introduce by an irrigation lumen and removed by an aspiration lumen.

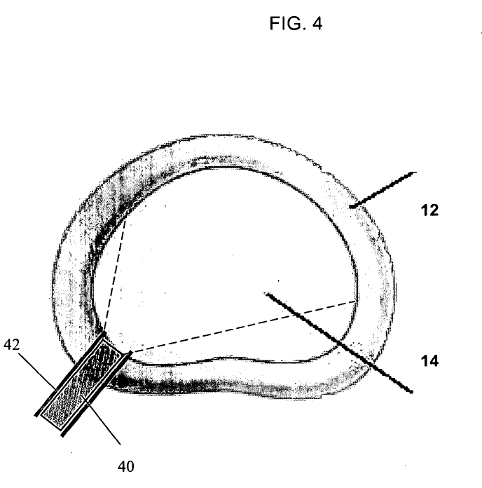

FIG. 4 provides a view of one embodiment of a method of visualizing an intervertebral disc. In the embodiment illustrated in FIG. 4, an access device, e.g., cannula, trocar, etc. is employed to provide access of the device to the internal body site, e.g., via a minimally sized incision. FIG. 4 shows a visualization device 40 according to an embodiment of the invention viewing the nucleus pulposus 12 of an intervertebral disc through an access port provided by an access device 42, such as a cannula.

Methods of invention may find use in any convenient application, including diagnostic and therapeutic applications. Specific applications of interest include, but are not limited to, intervertebral disc diagnostic and therapeutic applications. For example, methods of the invention include diagnostic applications, where a disc is viewed to determine any problems with the disc, if present. Methods of the invention also include treatment methods, e.g., where a disc is modified in some manner to treat and existing medical condition. Treatment methods of interest include, but are not limited to: annulotomy, nucleotomy, discectomy, annulus replacement, nucleus replacement, and decompression due to a bulging or extruded disc. Additional methods in which the imaging devices find use include those described in United States Published Application No. 20080255563

Methods and devices of the invention may be employed with a variety of subjects. In certain embodiments, the subject is an animal, where in certain embodiments the animal is a “mammal” or “mammalian.” The terms mammal and mammalian are used broadly to describe organisms which are within the class mammalia, including the orders carnivore (e.g., dogs and cats), rodentia (e.g., mice, guinea pigs, and rats), lagomorpha (e.g. rabbits), ungulates, e.g., horses, cows, goats, etc., and primates (e.g., humans, chimpanzees, and monkeys). In certain embodiments, the subjects (i.e., patients) are humans.

### Systems

Also provided are systems that include the imaging devices of the invention. Systems refer to configurations of components that include the devices of the invention, where the configurations are those present when the devices are ready to be used or in use. In addition to the imaging devices, embodiments of the systems may include control devices, e.g., that include programming which executes control instructions for the imaging devices and controls output of image data to a user. Furthermore, the systems may include access devices, guidewires, negative pressure sources, fluid reservoirs, power sources, etc., depending on the particular embodiment.

### Kits

Also provided are kits for use in practicing the subject methods, where the kits may include one or more of the above devices, and/or components of the subject systems, as described above. As such, a kit may include a visualization device, and may further include an access device, e.g., a cannula configured to be employed with the visualization device. The kit may further include other components, e.g., guidewires, stylets, etc., which may find use in practicing the subject methods. Various components may be packaged as desired, e.g., together or separately.

In addition to above mentioned components, the subject kits typically further include instructions for using the components of the kit to practice the subject methods. The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g. CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

### Computer Readable Storage Media

Also of interest is programming that is configured for operating a visualization device according to methods of invention, where the programming is recorded on physical computer readable media, e.g. any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. One of skill in the art can readily appreciate how any of the presently known computer readable mediums can be used to create a manufacture comprising a storage medium having instructions for operating a minimally invasive of the invention.

Programming of the invention includes instructions for operating a device of the invention, such that upon execution by the programming, the executed instructions result in execution of the imaging device to: illuminate a target tissue site, such as an intervertebral disc or portion thereof, with one of the white light source and the near-infra-red light source; and capture one or more image frames of the illuminated target tissue site with the imaging sensor. In certain embodiments, the executed instructions further result in capturing a white light video of the target tissue site made up of image frames obtained under white light illumination and capturing a near-infra-red light video of the target tissue site made up of image frames obtained under near-infra-red illumination. In certain embodiments, the executed instructions further result in producing a combined video made up of components of the white light video and near-infra-red video. In certain embodiments, the executed instructions further result in providing to a user a choice of viewing the white light video and the combined video.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

1. A minimally invasive imaging device, the imaging device comprising:
(a) an elongate member dimensioned to access an internal target tissue site and having a proximal and distal end;
(b) a white light source at the distal end;
(c) a near-infra-red light source the distal end; and
(d) an imaging sensor at the distal end.

2. The minimally invasive imaging device according to claim 1, wherein the white light source is a white light emitting diode.

3. The minimally invasive imaging device according to claim 1, wherein the white light source is an optical fiber operatively coupled to a white light emitter located at the proximal end of the elongate member.

4. The minimally invasive imaging device according to claim 1, wherein the near-infra-red light source is a near-infra-red light emitting diode.

5. The minimally invasive imaging device according to claim 1, wherein the near infra-red light source is an optical fiber operatively coupled to a near-infra-red light emitter located at the proximal end of the elongate member.

6. The minimally invasive imaging device according to claim 1, wherein the imaging sensor is a CMOS sensor or CCD sensor.

7. The minimally invasive imaging device according to claim 1, wherein the elongate member further comprises a tissue modifier.

8. The minimally invasive imaging device according to claim 1, wherein the tissue modifier is chosen from an electrode, mechanical cutting element, ultrasound transducer and laser.

9. The minimally invasive imaging device according to claim 1, wherein the elongate member further includes an irrigation lumen and an aspiration lumen.

10. The minimally invasive imaging device according to claim 1, wherein the elongate member is operatively coupled at the distal end to controller that is configured to alternate illumination of an intervertebral disc with the white light source and the near-infra-red source.

11. The minimally invasive imaging device according to claim 10, wherein the controller includes a processor configured to provide to a user a choice of viewing a white light video obtained solely under white light illumination or a combined video of a video obtained under white light illumination and a video obtained under near-infra-red illumination.

12. The minimally invasive imaging device according to claim 1, wherein the internal target tissue site is an intervertebral disc target site.

13. A method of visualizing an internal target tissue site, the method comprising:
(a) positioning a distal end of a minimally invasive intervertebral disc imaging device in viewing relationship to an internal target tissue site, where the imaging device comprises:
(i) an elongate member dimensioned to access the internal target tissue site and having a proximal end and a distal end;
(ii) a white light source at the distal end;
(iii) a near infra-red light source the distal end; and
(iv) an imaging sensor at the distal end; and
(b) illuminating the internal target tissue site with one of the white light source and the near-infra-red light source; and
(c) capturing one or more image frames of the illuminated internal target tissue site with the imaging sensor.

14. The method according to claim 13, wherein the method comprises sequentially illuminating the internal target tissue site with the white light source and the near-infra-red light source.

15. The method according to claim 14, wherein the method comprises capturing a white light video of the internal target tissue site made up of image frames obtained under white light illumination and capturing a near-infra-red light video of the internal target tissue site made up of image frames obtained under near-infra-red illumination.

16. The method according to claim 15, wherein the method comprises producing a combined video made up of components of the white light video and near-infra-red video.

17. The method according to claim 16, wherein the method comprises providing to a user a choice of viewing the white light video and the combined video.

18-23. (canceled)

24. An apparatus comprising a storage medium having instructions for operating a minimally invasive i imaging device comprising: (i) an elongate member dimensioned to access an internal target tissue site and having a proximal end and a distal end; (ii) a white light source at the distal end; (iii) a near-infra-red light source the distal end; and (iv) an imaging sensor at the distal end; wherein when executed by a computing platform, the executed instructions result in execution of the imaging device to:
(a) illuminate an internal target tissue site with one of the white light source and the near-infra-red light source; and
(b) capture one or more image frames of the illuminated internal target tissue site with the imaging sensor.

25. The apparatus according to claim 23, wherein the executed instructions further result in capturing a white light video of the internal target tissue site made up of image frames obtained under white light illumination and capturing a near-infra-red light video of the internal target tissue site made up of image frames obtained under near-infra-red illumination.

26. The apparatus according to claim 25, wherein the executed instructions further result in producing a combined video made up of components of the white light video and near-infra-red video.

27. The apparatus according to claim 25, wherein the executed instructions further result in providing to a user a choice of viewing the white light video and the combined video.
